(19) United States
(12) Patent Application Publication
(10) Pub. No.: US 2017/0224415 A1
(43) Pub. Date: Aug. 10, 2017

(54) RADIOFREQUENCY ABLATION CATHETER HAVING MESHED TUBULAR STENT STRUCTURE AND AN APPARATUS THEREOF

(71) Applicant: SHANGHAI GOLDEN LEAF MED TEC CO., LTD., Shanghai (CN)

(72) Inventors: Yonghua DONG, Shanghai (CN); Meijun SHEN, Shanghai (CN); Liang JI, Shanghai (CN); Zhengmin SHI, Shanghai (CN)

(21) Appl. No.: 15/501,662

(22) PCT Filed: Jun. 16, 2015
(86) PCT No.: PCT/CN2015/081584

§ 371 Date: Feb. 3, 2017

(30) Foreign Application Priority Data
Aug. 5, 2014 (CN) 201410381377.6
Oct. 17, 2014 (CN) 201410554508.6

(51) Int. Cl. A61B 18/14

(52) U.S. Cl. CPC A61B 18/1492; A61B 2018/00577

(57) ABSTRACT

A radiofrequency ablation catheter having a meshed tubular stent structure and an apparatus thereof, include a meshed tubular stent disposed at a front end of the catheter. The meshed tubular stent comprises and including a meshed tube (1). Both ends of the meshed tube are tapered to form a distal end and a proximal end of the meshed tubular stent. The intermediate segment of the meshed tubular stent has a contracted state and an expanded state. One or more electrodes (2) are fixed onto the intermediate segment. The radiofrequency ablation catheter has improved flexibility and provides great coverage for the blood vessels with different thicknesses and curves. When the meshed tubular stent expands in the blood vessels having different thicknesses of 4-12 mm, all of the electrodes (2) contact the walls. Moreover, when the meshed tubular stent expands in the curved blood vessels, all of the electrodes are ensured to contact the walls.

### FIELD OF THE INVENTION

The present invention relates to a radiofrequency ablation catheter, and more particularly to a radiofrequency ablation catheter having a meshed tubular stent structure and to a radiofrequency ablation apparatus including the radiofrequency ablation catheter described above.

### BACKGROUND OF THE INVENTION

In radiofrequency ablation systems, radiofrequency electrodes are key elements for contacting or approaching human tissue being treated and releasing radiofrequency energy. Radiofrequency electrodes are used for converting the radio frequency signal into the temperature field and for treating human tissues through thermal effects. During surgery, whether the radiofrequency electrodes effectively contact the wall has a decisive effect for the radiofrequency ablation treatment.

In the radiofrequency ablation catheter, the radiofrequency electrodes are mounted on the stent at the front end of the radiofrequency ablation catheter. The stent is used for carrying the radiofrequency electrodes, expanding and contacting the wall before the radiofrequency begins to be released, and contracting and retracting after the radiofrequency release is complete. Since the radiofrequency ablation is directly performed in the human blood vessels, the expansion dimension of the stent should fit the diameter of the human blood vessels.

The diameter of the human blood vessels varies from person to person, and there are also differences in the diameters of the blood vessels in the human body due to the differences of the different to-be-ablated sites. The diameters of most of the human blood vessels are ranged from about 2 to about 12 mm, and have large differences. In the conventional technique, the expansion dimension of the electrode end of a single radiofrequency ablation catheter is usually constant, cannot be adapted to the different diameters of the blood vessels in human bodies, and has narrow coverage for the human blood vessels having different diameters. Therefore, for the radiofrequency ablation operation in different patients, it is usually needed to change different specifications and types of the radiofrequency ablation catheters for performing ablation. Even so, in some situations, the radiofrequency electrode cannot still contact the wall at the same time during the surgery, thereby affecting the surgical results. Therefore, a new radiofrequency ablation catheter is needed, which has a special stent having effective expansion and adaptability to the blood vessels of different diameters, can be applied to the blood vessels of different diameters during the surgery, and ensures that a plurality of the electrodes contact the wall at the same time, thereby improving the coverage of the apparatus.

In addition, the adaptability of the conventional radiofrequency ablation catheter to the curved blood vessels are generally poor. The electrodes of most of the radiofrequency ablation catheters in the curved blood vessels cannot effectively contact the wall. Hence, if a new radiofrequency ablation catheter can also improve the coverage for the curved blood vessels, the application range of the radiofrequency ablation will be greatly broadened, the effect of the radiofrequency ablation will be improved at the same time, and there will be a positive effect on the promotion of radiofrequency ablation.

### SUMMARY OF THE INVENTION

The primary technical problem to be resolved by the present invention is to provide a radiofrequency ablation catheter having a meshed tubular stent structure, which has excellent adaptability to the blood vessels having different diameters and the curved blood vessels, and has wide coverage.

Another technical problem to be resolved by the present invention is to provide a radiofrequency ablation apparatus including the radiofrequency ablation catheter described above.

In order to achieve the aforementioned object, the following technical solutions are adopted in the present invention:

A radiofrequency ablation catheter having a meshed tubular stent structure includes a meshed tubular stent disposed at a front end of the catheter and including a meshed tube, wherein both ends of the meshed tube are tapered to form a distal end and a proximal end of the meshed tubular stent, an intermediate segment of the meshed tubular stent has a contracted state and an expanded state, and one or more electrodes are fixed on at least one filament of the intermediate segment of the meshed tubular stent.

Preferably, before assembly, the meshed tube is shaped to have an intermediate cylinder, both ends of which are tapered; and after assembly, the meshed tube is shaped into a cylinder.

Alternatively, before assembly, the meshed tube is shaped into a cylinder; and after assembly, the meshed tube is shaped into a round drum body which has an intermediate convex and both naturally tapered ends.

Preferably, the radiofrequency ablation catheter further includes a radiofrequency line and a thermocouple wire disposed inside each of the electrodes; wherein the radiofrequency line, the thermocouple wire and the filament are independent wire materials; or a portion of the filament has a function of the radiofrequency line; or the radiofrequency line and the thermocouple wire are made into one wire.

Preferably, axial projections of a plurality of the electrodes in an axial direction of the meshed tubular stent do not overlap each other.

Preferably, a plurality of the electrodes are arranged in a straight line or staggered in a plurality of straight lines on an expansion diagram of a circumferential surface of the meshed tube.

Preferably, both ends of the meshed tube are provided with a first connecting tube and a second connecting tube; the meshed tubular stent further includes a central drawing filament disposed along a central axis thereof, wherein one end of the central drawing filament is fixed on the first connecting tube disposed at the distal end of the meshed tubular stent, or the central drawing filament penetrates through the first connecting tube and is confined outside the first connecting tube; the other end of the central drawing filament penetrates through an inside of the meshed tubular stent and then through a center of the second connecting tube disposed at the proximal end of the meshed tubular stent; the central drawing filament is configured to axially draw the meshed tubular stent relative to the second connecting tube, and the central drawing filament is configured to slide toward the distal end of the meshed tubular stent relative to the second connecting tube.

Preferably, the proximal end of the meshed tubular stent is connected to a multi-hole tube, wherein one end of the central drawing filament is fixed on the distal end of the meshed tubular stent, or the central drawing filament is confined outside the distal end of the meshed tubular stent, and thus configured to freely slide relative to the distal end of the meshed tubular stent; wherein the other end of the central drawing filament penetrates through a central hole of the multi-mole tube; a radiofrequency line, a thermocouple wire, and the filament are disposed inside each of the electrodes; both ends of the electrodes are fixed on the meshed tubular stent; one end of the thermocouple wire and one end of the radiofrequency line are fixed inside the electrode; and the other end of the thermocouple wire and the other end of the radiofrequency line penetrate through a corresponding hole in the multi-hole tube and then are connected to an external device.

Preferably, an opening is disposed on a circumference of each of the electrode.

Preferably, the meshed tube is woven and formed by the single filament or a plurality of the filaments; or the meshed tube is processed and formed by a metal material or a polymer material.

A radiofrequency ablation apparatus includes a radiofrequency ablation catheter as described above, and a control handle and a radiofrequency ablation main machine, both connected to the radiofrequency ablation catheter.

A radiofrequency ablation catheter having a meshed tubular stent structure is provided in the present invention, and radiofrequency electrodes are disposed on the meshed tubular stent. The meshed tubular stent has excellent flexibility, so that when the meshed tubular stent is expanded and drawn in the blood vessels having different thicknesses, all of the electrodes contact the wall. Moreover, by arranging a plurality of the electrodes disposed on the meshed tubular stent, the electrodes do not to overlap in the axial direction of the meshed tubular stent, thereby not causing excessive ablation. The meshed tubular stent has improved flexibility and wide coverage for the blood vessels having different diameters, which can meet the requirements of the radiofrequency ablation for the blood vessels of at least 4-12 mm. Moreover, the meshed tubular stent also has effective coverage for the curved blood vessels.

### BRIEF DESCRIPTION OF THE DRAWINGS

FIG. 1 is a schematic structural diagram of a meshed tubular stent in accordance with a first embodiment of the present invention.

FIG. 2a is a structural schematic diagram of a cylindrical meshed tube having 12 filaments in a cross section.

FIG. 2b is a cross-sectional schematic diagram of a cylindrical meshed tube having 12 filaments in a cross section of FIG. 2a.

FIG. 3a is a structural schematic diagram of a cylindrical meshed tube having 18 filaments in a cross section.

FIG. 3b is a cross-sectional schematic diagram of a cylindrical meshed tube having 18 filaments in a cross section of FIG. 3a.

FIG. 4 is a schematic diagram of axial projections of 6 electrodes without overlapping distribution in an axial direction of the meshed tubular stent.

FIG. 5 is a schematic diagram of circumferential projections of 6 electrodes evenly distributed on a circumferential cross section of the meshed tubular stent.

FIG. 6 is a structural schematic diagram of 6 electrodes disposed on a meshed tube including 12 filaments in a cross section.

FIG. 7 is a structural schematic diagram of 6 electrodes disposed on a meshed tube including 18 filaments in a cross section.

FIG. 8 is a structural schematic diagram of 6 electrodes disposed on a meshed tube including 24 filaments in a cross section.

FIG. 9 is a working principle diagram showing a meshed tubular stent contacting the wall in a thin blood vessel

FIG. 10 is a cross-sectional schematic diagram of a meshed tubular stent as shown in FIG. 9.

FIG. 11 is a working principle diagram showing a meshed tubular stent contacting the wall in a thick blood vessel.

FIG. 12 is a structural schematic diagram of a meshed tube shaped into a cylinder in the second embodiment.

FIG. 13 is a structural schematic diagram of the assembled meshed tubular stent in a round drum shape in the second embodiment.

FIG. 14a, FIG. 14b, FIG. 14c, and FIG. 14d are respectively experimental result diagrams of the same meshed tubular stent expanded in the simulative blood vessels having the diameters of 4 mm, 6 mm, 8 mm, and 12 mm, and contacts the walls thereof, wherein the simulative blood vessel in FIG. 14b has radians.

FIGS. 15a and 15b are experimental result diagrams of the meshed tubular stent, which respectively automatically expands and is drawn to contact the wall, in the same thick simulative blood vessels.

### DETAILED DESCRIPTION OF THE PREFERRED EMBODIMENTS

The technical contents of the present invention are described in detail with reference to the accompanying drawings and the specific examples. For convenience, the end close to the operator (away from the ablation site) is referred to as the proximal end, and the end away from the operator (close to the ablation site) is referred to as the distal end.

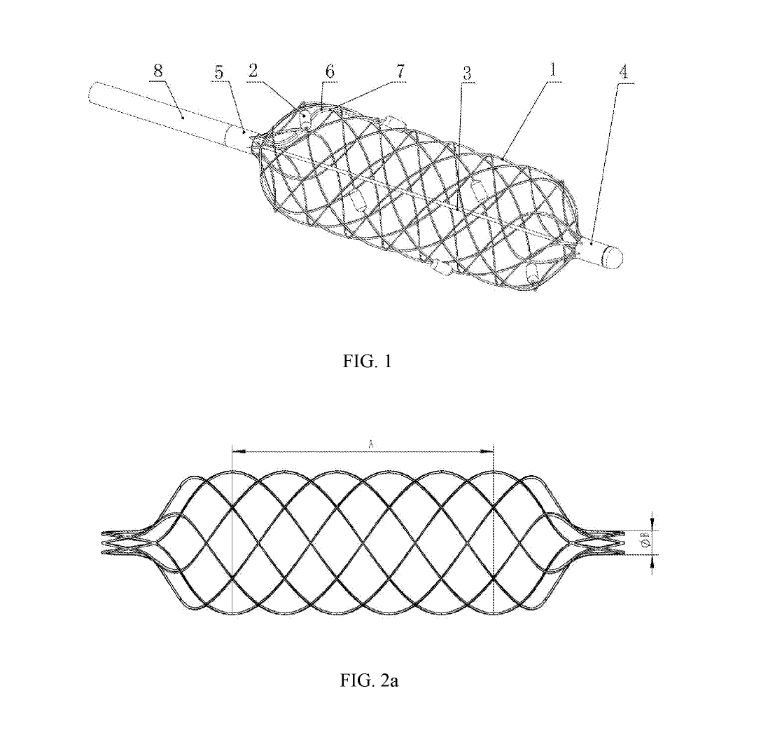

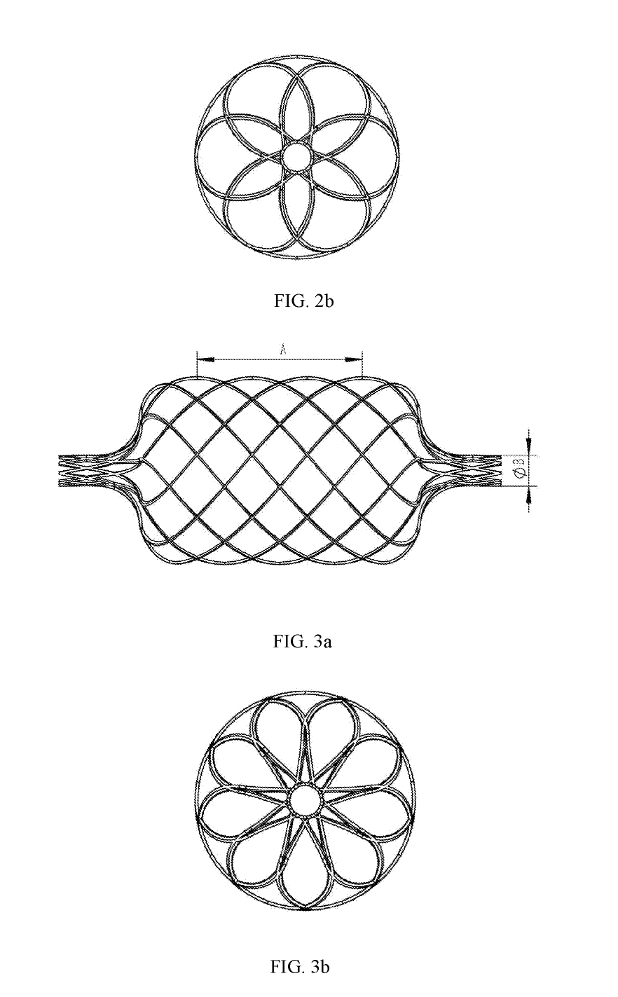

As shown in FIG. 1, the front end of the radiofrequency ablation catheter provided in the present invention has a meshed tubular stent, which includes a meshed tube 1. The meshed tube 1 is woven and formed by one single filament or a plurality of filaments. The meshed tube 1 is processed and formed by a metal material or a polymer material. Specifically, the meshed tube 1 is obtained using polymer materials or metal materials by various processing means, such as carving, machining, powder metallurgy, injection molding or 3D printing. The meshed tube 1 may be shaped or may not be shaped before assembly. The meshed tube 1 can be deformed during assembly and expansion. After assembly, both ends of the meshed tube 1 are tapered to form the distal end and proximal end of the meshed tubular stent. Connecting pipes 4 and 5 are respectively disposed on the tapered ends of the meshed tube 1. An intermediate segment of the meshed tubular stent has a contracted state and an expanded state. One or more electrodes 2 are fixed on at least one filament of the intermediate segment (the area as shown in FIG. 2) of the meshed tubular stent 1. The intermediate segment of the meshed tube 1 expands and contacts the wall in the lumen on the ablation site. Furthermore, in order to ensure weaving density and flexibility of the meshed tubular stent, the number of the filaments in the cross section of the meshed tube 1 is preferably limited to fewer than 30.

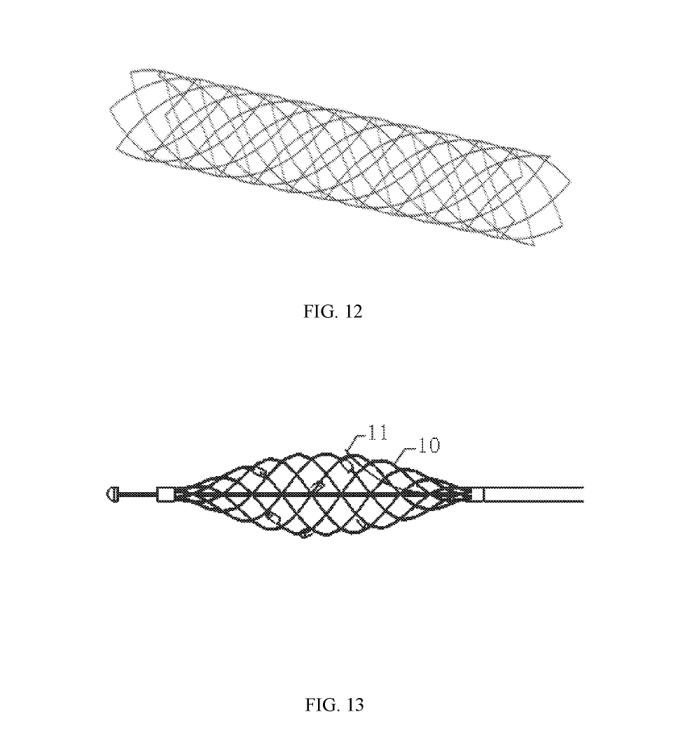

In the following, two meshed tubular stents, in which a meshed tube 1 first undergoes a shaping process and subsequently is assembled, are taken as examples. The structures of the meshed tubular stents of the radiofrequency ablation catheters provided in the present invention and the contact thereof with the walls are described. In the first embodiment, before the meshed tubular stent is assembled, the meshed tube is shaped to have an intermediate cylinder, the both ends of the meshed tube are tapered, and the intermediate segment and the ends are connected at an oblique angle between 10° and 90°, and have arc transition, as shown in FIG. 2. After the assembly, the overall shape of the meshed tubular stent is a cylinder, as shown in FIG. 1. In the second embodiment, before the meshed tubular stent 10 is assembled, the meshed tube is shaped into a cylinder, as shown in FIG. 12, and both ends of the meshed tube are not tapered. During assembly, both ends are tapered by connectors. Subsequently, the meshed tube is shaped into a round drum body, which has an intermediate convex and both naturally tapered ends. The two embodiments are described in detail below.

### First Embodiment

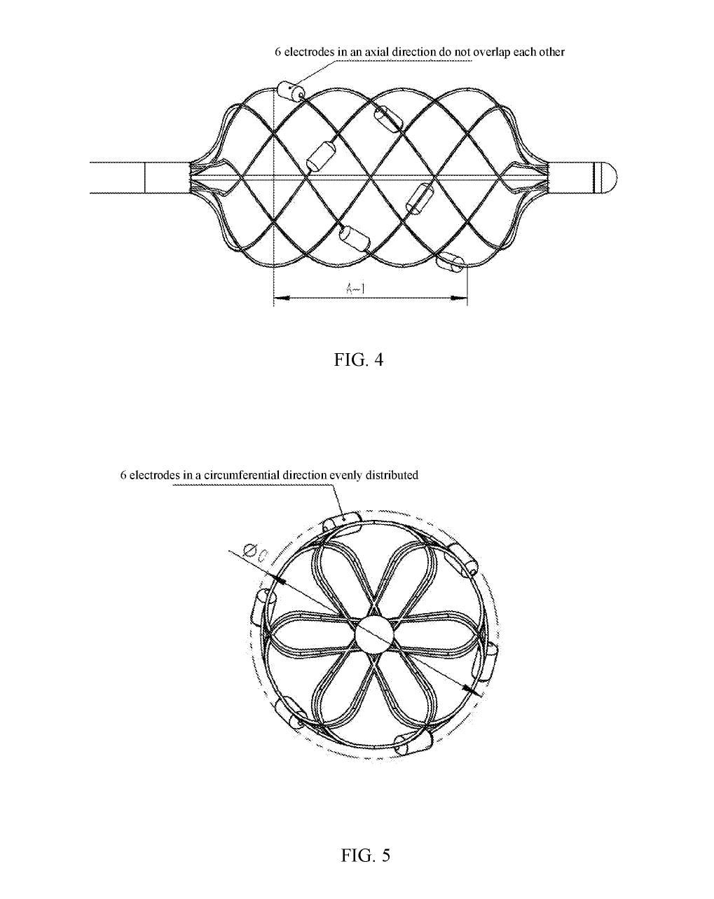

As shown in FIG. 2a and FIG. 3a, in the first embodiment, before the meshed tubular stent is assembled, the meshed tube is shaped into an intermediate cylinder, and both ends of the meshed tube are tapered, as shown in FIG. 2. Specifically, a transition zone at a specific oblique angle is disposed between the intermediate cylindrical segment and the both tapered segments. Preferably, the both ends of the transition zone are connected with the cylindrical segment and the tapered segments through arc transition. The diameter of the tapered segment is equivalent to the diameter of the ablation catheter. When being assembled, the both tapered segments of the meshed tube 1 are respectively fixed in a first connecting tube 4 and a second connecting tube 5, so that the overall shape of the meshed tubular stent after assembly presents a cylinder as shown in FIG. 1.

FIG. 2a, FIG. 2b, FIG. 3a, and FIG. 3b are structural schematic diagrams of the cylindrical meshed tubes 1, the cross sections of which respectively include 12 filaments and 18 filaments. Comparing the two structures, it can be seen that when the number of filaments in the cross sections of the cylindrical meshed tubes 1 increases, the length of the filaments between adjacent nodes suitably reduces. The length of the meshed tubular stent ensures the suitable number of electrodes arranged in the intermediate segment while also ensuring suitably shortening the length of the meshed tubular stent on the basis of the sufficient flexibility in the blood vessels of 2-10 mm.

The radiofrequency ablation catheter also includes thermocouple wires 6 and radiofrequency lines 7 disposed inside each electrode 2. When meshed tube 1 is woven using a single filament, a single nickel-titanium filament, a stainless steel filament, or other filamentary material (e.g., medical polymer material) can be used to independently weave a scaffold, and the thermocouple wires 6 and the radiofrequency lines 7 are disposed on the scaffold. The filaments, the radiofrequency lines 7 and the thermocouple wires 6 can be separate wire materials, and the thermocouple wire 6 and the radiofrequency lines 7 are respectively wound with the meshed tubular stent. The mesh filaments, the radiofrequency lines 7 and the thermocouple wire 6 have their own functions. Alternatively, the thermocouple wires 6 and the radiofrequency lines 7 can also be made into a single wire. The radiofrequency line 7 and thermocouple wire 6 are integrated, and subsequently wounded with the meshed tubular stent.

When the meshed tube 1 is woven using multiple filaments, the meshed tube 1 can be directly woven using multiple filaments as described above, and the thermocouple wire 6 and the radiofrequency line 7 are disposed on the meshed tube 1, or some of the filaments (i.e. the mesh filaments used for fixing the electrodes 2) are replaced with the radiofrequency lines 7 (or the same wire materials including the radiofrequency lines 7 and the thermocouple wires 6), so that some of the filaments have the function of the radiofrequency lines, and the meshed tube 1 is woven and formed using the multiple radiofrequency lines 7 and the remaining multiple filaments together. When the meshed tube 1 is woven using multiple radiofrequency lines 7 and multiple filaments, after the meshed tube 1 is woven, the thermocouple wires 6 can be wound with the radiofrequency line 7, and the multiple electrodes 2 are fixed to the radiofrequency lines 7 in the meshed tube 1. Certainly, when the meshed tube 1 is woven using multiple filaments, multiple radiofrequency lines 7 and multiple filaments may be wound together as a single braided wire, and the meshed tube 1 is woven and formed using the aforementioned braided wires and other filaments together, that is to say, the meshed tubular stent is not limited to the structure of the meshed tube woven by a single filament, and other structural alternations are possible.

In the practical manufacture of the meshed tubular stent, it is required for each mesh filament (or radiofrequency line) to be insulated. An insulation layer is directly formed on the mesh filament. Alternatively, after the electrodes are fixed on the mesh filaments, the rest parts of the filaments excluding the electrodes are insulated. One or more electrodes may or may not be fixed on each filament used to form the meshed tubular stents. For example, when the meshed tube, whose cross section includes 24 filaments, is woven using 12 filaments, by respectively disposing an electrode on 6 filaments thereof, the meshed tubular stent having a high strength can be formed, and the distribution of 6 electrodes on the meshed tubular stent does not cause excessive ablation. For another example, when the meshed tube whose cross section includes 6 filaments is woven using 2 filaments, by respectively disposing 6 electrodes on each filament, the meshed tubular stent in which 12 electrodes are evenly distributed on the outer surface of the meshed tube. For preventing the electrodes from excessively ablating the walls of the blood vessels, during disposing the multiple electrodes on the meshed tubular stent, the projections of a plurality of the electrodes in the axial direction of the meshed tubular stent preferably do not overlap each other.

FIG. 4 and FIG. 5 are schematic diagrams of 6 electrodes disposed on the meshed tube 1 in a meshed tubular stent provided in the present invention. Six electrodes disposed on the cylindrical meshed tube 1 are taken as an example for description, herein. In the following description, the number of the braided filaments in the cross section of the meshed tube is only described as a reference, and the specific number of the braided filaments is not taken into account. In the meshed tubular stent provided in the present invention, six electrodes 2 are disposed on the circumferential surface of the intermediate segment. As shown in FIG. 4, it can be seen that when the meshed tubular stent is expanded, the axial projections of the six electrodes 2 in the axial direction of the meshed tubular stent do no overlap. As shown in FIG. 5, it can be seen that when the meshed tubular stent is expanded, the circumferential projections of the six electrodes 2 are evenly distributed over the circumferential cross section of the meshed tubular stent. Although the arrangement pattern of a plurality of the electrodes is arranged in a spiral form on the circumferential surface of the meshed tubular stent in this embodiment, this does not mean that the arrangement pattern of a plurality of electrodes requires a specific shape. For ensuring a plurality of the electrodes contacting the wall at the same time and ensuring the ablation effect, the axial projections of the electrodes on the meshed tubular stent do not overlap each other, so that when the meshed tubular stent expands in the blood vessels, regardless of the thickness of the blood vessels, no electrode causes excessive ablation to the blood vessels, and any damage to the blood vessels is prevented.

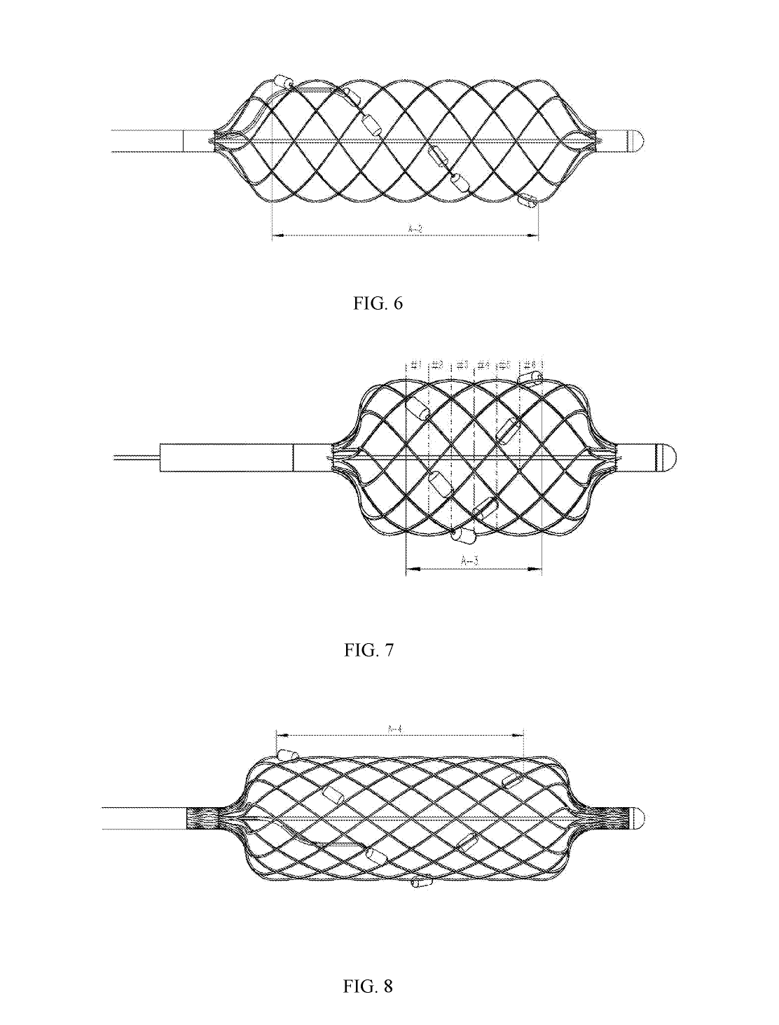

FIG. 6, FIG. 7, and FIG. 8 are schematic diagrams of 6 electrodes disposed on the circumferential surface when the meshed tube 1 includes 12 filaments, 18 filaments, and 24 filaments in a cross section in the first embodiment provided by the present invention. According to the order from upper left to lower right, six electrodes 2 in the expansion diagram of the meshed tube 1 are labeled from electrode #1 to electrode #6. In the embodiment as shown in FIG. 6, the six electrodes are staggered in a broken line consisting of 2 straight lines on the expansion diagram of the circumferential surface of the meshed tube 1 including 12 filaments on the cross section thereof. In the embodiments as shown in FIG. 7 and FIG. 8, 6 electrodes are distributed from upper left to lower right on the expansion diagram of the circumferential surface of the meshed tube 1 including 18 or 24 filaments on the cross section thereof, and arranged in a straight line, so that in the above 3 embodiments, the six electrodes are arranged in a spiral shape on the circumferential surface of the meshed tube. Although in the accompanying figures provided in the present application, six electrodes are regularly arranged on the circumferential surface of the meshed tubular stent, this does not mean that it is required for a plurality of electrodes to be regularly arranged on the circumferential surface of the meshed tubular stent. In the embodiments without providing the specific structural diagrams, a plurality of electrodes can also be irregularly arranged on the circumferential surface. Certainly, a plurality of electrodes can also be arranged in other shapes. In the practical ablation operation, according to the location of a single electrode the nerve tissue in the vicinity thereof is ablated. The case where the electrodes are disposed on the meshed tube in the round drum body is similar, and the details are not described redundantly in the second embodiment.

As shown in FIG. 1, the both tapered ends of the meshed tubular stent provided in the present invention are provided with a first connecting tube 4 and a second connecting tube 5. The first connecting tube 4 is disposed at a distal end of the meshed tubular stent, and the second connecting tube 5 is disposed at a proximal end of the meshed tubular stent. For successfully disposing the electrodes 2 on the filaments of the meshed tube 1, the center of the electrode 2 provided in the present invention is provided with a round hole, and the circumference of the electrode 2 is provided with an opening. When the center of the electrode 2 is provided with a round hole, the weaving of the meshed tube is completed after the electrodes are fixed on the filaments. Moreover, since the internal space thereof is large, it is relatively easy to fix the thermocouple wire 6 and radiofrequency line 7 in the interior thereof during assembly. When the circumference of the electrode 2 is provided with an opening, it is convenient to engage the electrodes 2 with the assembled meshed tube 1, and fix the both ends of the electrodes 2 onto the filaments for completing the disposition of the electrodes 2. The electrodes 2 are disposed in the directions which are consistent with the directions in which the filaments extend, so the directions generally are not parallel to the axis of the meshed tubular stent and are inclined at angles to the axis. The inclination angles of the electrodes 2 vary during the contraction or the expansion of the meshed tubular stent. When the meshed tubular stent contracts, the inclination angles decrease, and when the meshed tubular stent expands, the inclination angles increase and gradually approach the vertical direction.

In addition, for controlling the contraction or the expansion of the meshed tubular stent in the blood vessels, a central drawing filament 3 is also disposed in the meshed tubular stent. In the first embodiment, one end of the central drawing filament 3 is fixed on the first connecting tube disposed at the distal end of the meshed tubular stent, and the other end penetrates through the inside of the meshed tubular stent and then through the second connecting tube 5 disposed at the proximal end of the meshed tubular stent. Moreover, the central drawing filament 3 extends through the central hole of the multi-hole tube 8 connected with the proximal end of the meshed tubular stent to the control handle disposed at the end of the catheter. The central drawing filament 3 is configured to draw the meshed tubular stent in the axial direction relative to the second connecting tube 5 and the multi-hole tube 8 under an external force. When the meshed tubular stent in the blood vessel is compressed by the wall of the blood vessel to undergo contractive deformation, the central drawing filament 3 automatically slides, the length of the meshed tube 1 is lengthened, and the outer diameter is reduced. When the central drawing filament 3 is drawn back from the outside of the catheter, the meshed tubular stent expands, the length of the meshed tube 1 is shortened, and the outer diameter is increased, so that a plurality of electrodes contact the wall of the blood vessel having a large diameter. When the central drawing filament 3 is pushed forward from the outside of the catheter, the meshed tubular stent contracts, thereby moving the location of the meshed tubular stent within the blood vessel or withdrawing the meshed tubular stent from the blood vessel to the outside of the body. During the movement, damage caused by the meshed tubular stent to the walls of the blood vessels is avoided.

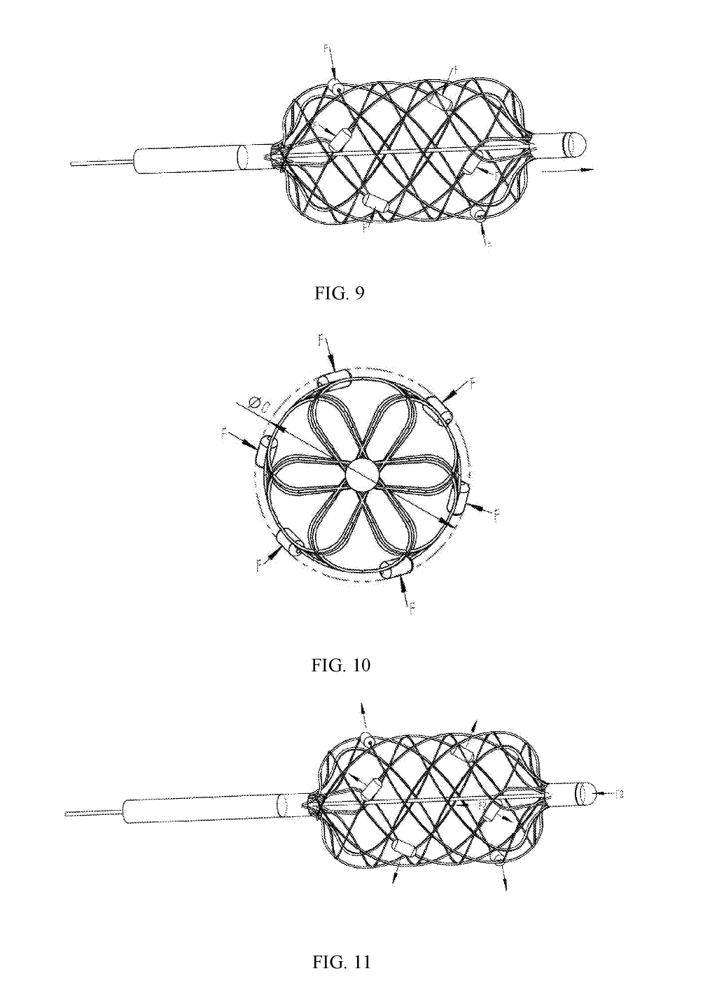

The flexibility of the meshed tubular stent in the first embodiment provided by the present invention is now described with reference to FIGS. 9, 10, and 11.

As the collapsed meshed tubular stent after protruding from the sheath naturally expands, as shown in FIG. 1, it is assumed that an initial outer diameter of the naturally expanding meshed tubular stent is C mm. As shown in FIG. 9 and FIG. 10, when the diameter of the to-be-ablated blood vessel is smaller than C mm, the meshed tubular stent is squeezed by the wall of the blood vessel during the natural dilation and is in a squeezed state. In this case, the length of the meshed tube 1 is lengthened, the distal end thereof moves forward in the blood vessel, each electrode 2 in the blood vessel completely contacts the wall under the effect of the pressure F from the wall of the blood vessel, and the contact condition is effective. When the diameter of the to-be-ablated blood vessel is greater than or equal to C mm, the meshed tubular stent after the natural expansion does not completely contact the wall. As shown in FIG. 11, when the central drawing filament 3 is drawn outward by applying the drawing force F2, the length of the meshed tubular stent is reduced, and the meshed tube 1 is expanded and in the expanded state. During this process, the electrodes 2 move toward and gradually contact the wall of the blood vessel, and finally effectively contact the wall of the blood vessel.

Furthermore, the radiofrequency ablation catheter further includes a multi-hole tube 8. The multi-hole tube 8 is connected with the proximal end of the meshed tubular stent (i.e., connected with the second connecting tube 5). One end of the central drawing filament 3 disposed in the meshed tubular stent is fixed at the distal end of the meshed tubular stent. The other end penetrates through the proximal end of the meshed tubular stent and the central hole of the multi-mole tube 8, extends to the outside of the catheter and is connected with the control handle. A thermocouple wire 6, a radiofrequency line 7, and a filament are disposed inside each electrode 2, both ends of the electrodes 2 are fixed on the filaments of the meshed tube, one end of the thermocouple wire and one end of the radiofrequency line are fixed inside the electrode 2, and the other end of the thermocouple wire 6 and the other end of the radiofrequency line 7 penetrate through a corresponding hole in the multi-hole tube 8 and then are connected to an external device. Since the coverage of the meshed tubular stent for the blood vessels having different diameter is improved, the same radiofrequency ablation catheter having the aforementioned meshed tubular stent can be used for radiofrequency ablation in different patients.

Meanwhile, the meshed tubular stent provided in the present invention has excellent adaptability to the curved blood vessels. After the meshed tubular stent is expanded and contacts the wall of the curved blood vessel, the whole meshed tubular stent is configured to be bent and adapted to the shape of the blood vessel. A plurality of electrodes disposed on the intermediate segment simultaneously contact the wall. In this embodiment, the effect of contact with the wall of the curved blood vessel is not shown, but the adaptability of the meshed tubular stent provided by the present invention can be understood in accompaniment with the effect diagram of the second embodiment.

### Second Embodiment

As shown in FIG. 12 and FIG. 13, in the first embodiment, before the meshed tubular stent is assembled, the meshed tube is shaped to have an intermediate cylinder, and the both ends of the meshed tube are tapered, as shown in FIG. 2. Specifically, a transition zone at a specific oblique angle is disposed between the intermediate cylindrical segment and the both tapered segments. Preferably, the both ends of the transition zone are connected with the cylindrical segment and the tapered segments through arc transitions. The diameter of the tapered segment is equivalent to the diameter of the ablation catheter. When being assembled, the both tapered segments of the meshed tube 1 are respectively fixed in a first connecting tube 4 and a second connecting tube 5, so that the overall shape of the meshed tubular stent after assembly presents a cylinder as shown in FIG. 1. The shape of the meshed tube and the shape thereof after assembly in the present embodiment are different from those in the first embodiment. Before assembly, the meshed tube of the meshed tubular stent is shaped into a cylinder, and both ends of the meshed tube are not tapered in advance, so that after the first connecting tube and the second connecting tube are assembled on the both ends of the meshed tube, the overall shape of the meshed tubular stent 10 presents a round drum body with an intermediate segment shaped into a convex and the both ends naturally tapered. After the meshed tubular stent 10 is expanded in the blood vessel and contacts the wall, a plurality of electrodes 2 distributed in the intermediate segment in the meshed tubular stent 10 simultaneously contact the wall of the blood vessel. Moreover, since the meshed tube having a round drum body is squeezed by the wall of the blood vessel during the expansion process, the contact effect of a plurality of electrodes 2 is improved.

In this embodiment, the central drawing filament is disposed in a manner different from that in the first embodiment. As shown in FIG. 13, one end of the central drawing filament is not fixed onto the first connecting tube, but penetrates through the first connecting tube, and is connected to the tip of the radiofrequency ablation catheter, thereby being confined to the outside of the first connecting tube (e.g. the distal end of the meshed tubular stent). The other end of the central drawing filament penetrates through the interior of the meshed tubular stent and extends out of the center of the second connecting tube. Therefore, in this embodiment, the central drawing filament is configured to draw the meshed tubular stent in an axial direction in relative to the second connecting tube, and the central drawing filament is configured to freely slide toward the distal end of the meshed tubular stent in relative to the first connecting tube and the second connecting tube.

Moreover, in the second embodiment, as shown in FIG. 13, a central puncture needle 11 is also disposed in the meshed tubular stent 10. The central puncture needle 11 protrudes from the surface of the meshed tube and penetrates into the wall of the blood vessel when the meshed tubular stent 10 is expanded and contacts the wall. In this embodiment, the central puncture needle 11 is drawn back inside the meshed tubular stent 10 when the meshed tubular stent 10 is contracted. Certainly, a similar puncture needle may also be disposed in the first embodiment.

Since the shape of the meshed tube before assembly and the arrangement of the central drawing filament in the second embodiment are different from those of the meshed tube in the first embodiment, and the rest of the configurations are the same as those in the first embodiment, the specific configurations are not described in detail herein. In the following, the flexibility of the meshed tubular stent provided in the second embodiment in the blood vessels having different diameters and in the curved blood vessels is described based upon the specific simulation experiment.

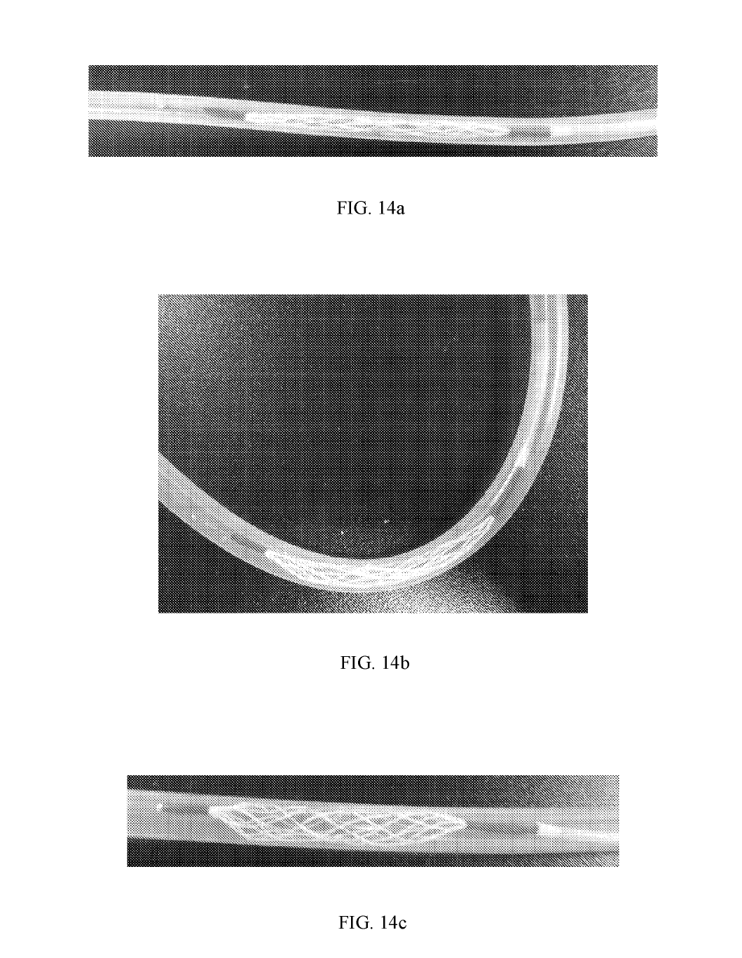

FIG. 14a, FIG. 14b, FIG. 14c, and FIG. 14d are respectively experimental result diagrams after the same meshed tubular stent of a radiofrequency ablation catheter protrudes from a sheath, expands in the simulative blood vessels having diameters of 4 mm, 6 mm, 8 mm, and 12 mm, and contacts the walls thereof, wherein the simulative blood vessel in FIG. 14b has radians. As shown in FIG. 14a-FIG. 14d, it can be seen that the same meshed tubular stent effectively contacts the walls of the blood vessels having different diameters, has excellent adaptability, and has excellent coverage for the blood vessels having different diameters. Moreover, as shown in FIG. 14b, it can be seen that the meshed tubular stent also has excellent adaptability for curved blood vessels. Thus, in the practical radiofrequency surgery, there is no specific requirement of the radiofrequency ablation catheter for the shape of the blood vessels on the ablated site, thereby overcoming the limitations of the conventional radiofrequency ablation catheters.

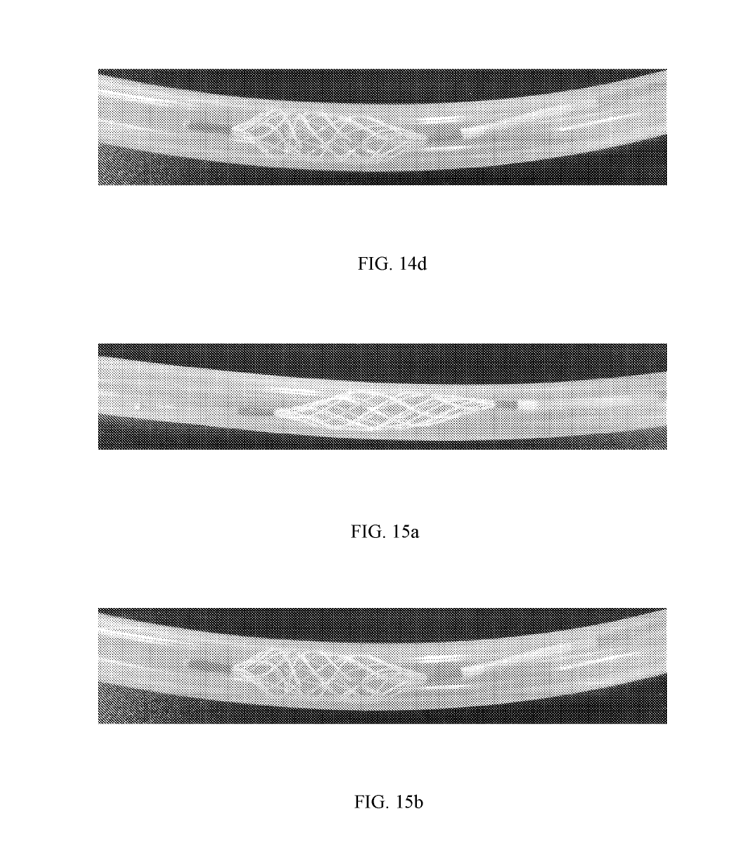

When the meshed tubular stent is expanded within the thin blood vessel, a plurality of electrodes disposed on the intermediate segment ensure the effective contact with the wall during the natural expansion process, as shown in FIG. 14a. When a meshed tubular stent is expanded within thick the blood vessel, typically, for example, after the natural expansion in the blood vessel having the diameter of 12 mm, as shown in FIG. 14d, since the initial outer diameter of the meshed tubular stent is smaller than the diameter of the blood vessel, most of the electrodes on the meshed tube cannot contact the wall, as shown in the condition diagram in FIG. 15a. The effective condition of a plurality of electrodes contacting the wall is ensured by drawing the central drawing filament.

It is explained herein that FIG. 14a-FIG. 15b are the experimental result diagrams in actual simulation experiments. In order to more faithfully reflect the flexibility of the meshed tubular stent of the radiofrequency ablation catheter provided in the present invention and the adaptability to the curved blood vessel. When submitting the present application, the applicant provides the actual effect diagram, without providing the corresponding line drawings. Earnestly request the examiner's understanding.

### Third Embodiment

In the meshed tubular stent provided in the first embodiment and the second embodiment, the meshed tube undergoes a shaping process before assembly. In the third embodiment provided in the present invention, the meshed tube does not undergo any specific shaping process before the meshed tube is assembled into the meshed tubular stent. When the radiofrequency ablation catheter protrudes from the sheath, the meshed tubular stent cannot expand spontaneously, but it is ensured that a plurality of electrodes disposed on the intermediate segment simultaneously contact the wall by drawing the central drawing filament. Furthermore, after the meshed tubular stent is expanded and contacts the wall, the axial projections of a plurality of electrodes in the axial direction of the meshed tubular stent do not overlap each other, and the circumferential projections of a plurality of electrodes are evenly distributed over the circumferential cross section of the meshed tubular stent.

The radiofrequency ablation catheter provided in the present invention is described above. The present invention also provides a radiofrequency ablation apparatus including the radiofrequency ablation catheter described above. In addition to the radiofrequency ablation catheter described above, the radiofrequency ablation apparatus includes a control handle and a radiofrequency ablation catheter main machine, both connected to the radiofrequency ablation catheter. The central drawing filament in the meshed tubular stent is connected to the control handle through the multi-hole tube, and the control handle may control the radiofrequency ablation catheter to move forward, move backward, and turn. The radiofrequency lines and the thermocouple wires in the meshed tubular stent are connected to the corresponding circuit in the radiofrequency ablation catheter main machine respectively via the multi-hole tube, thereby realizing the radiofrequency control and the temperature monitoring of the radiofrequency ablation catheter main machine for a plurality of electrodes. The setting of the control handle and the setting of the radiofrequency ablation catheter main machine can be seen in the patents previously applied for and filed by the applicant, and the specific structure thereof is not described in detail herein.

In actual clinical treatment, the radiofrequency ablation catheter and the radiofrequency ablation apparatus provided in the present invention can be applied to nerve ablation in different parts, the blood vessels, or the trachea having different diameters: for example, nerve ablation in the renal artery for treating patients with refractory hypertension, nerve ablation in the celiac artery for treating patients with diabetes, for example, the ablation of the tracheal / bronchial vagal nerve branch for treating patients with asthma, the ablation of the duodenum vagus nerve branch for treating patients with duodenal ulcer, and, in addition, nerve ablation in other blood vessels in the renal pelvis, the pulmonary artery or the trachea. It should be noted that the radiofrequency ablation catheter provided in the present invention is not limited to the aforementioned applications in clinical treatments, but can also be used for nerve ablation at other sites.

In summary, since in the radiofrequency ablation catheter provided in the present invention, a meshed tube woven by a single filament or multiple filaments is used, and the electrodes, which have a plurality of arrangement forms in the expanded state to meet specific requirements, are disposed on the circumferential surface of the meshed tube, when the meshed tubular stent is expanded in the blood vessels having different diameters, a plurality of electrodes all effectively contact the wall. The meshed tubular stent has improved flexibility and wide coverage for the blood vessels having different diameters, which can meet the requirements of the radiofrequency ablation for the blood vessels of at least 4-12 mm. Moreover, the meshed tubular stent also has effective coverage for the curved blood vessels. Therefore, the radiofrequency ablation catheter provided in the present invention and the radiofrequency ablation apparatus including the aforementioned radiofrequency ablation catheter have wide coverage for nerve ablation operations in different patients.

The radiofrequency ablation catheter having meshed tubular stent structure and the device thereof provided by the present invention have been described in detail. A person of ordinary skill in the art who makes any obvious change to this invention without departing from the substantial spirit of the present invention will commit a violation of the patent rights of this prevent invention, and will take the corresponding legal responsibilities.

1. A radiofrequency ablation catheter having a meshed tubular stent structure, comprising:
a meshed tubular stent disposed at a front end of the catheter and including a meshed tube, wherein both ends of the meshed tube are tapered to form a distal end and a proximal end of the meshed tubular stent, an intermediate segment of the meshed tubular stent has a contracted state and an expanded state, and one or more electrodes are fixed on at least one filament of the intermediate segment of the meshed tubular stent.

2. The radiofrequency ablation catheter as claimed in claim 1, wherein before assembly, the meshed tube is shaped to have an intermediate cylinder, both ends of which are tapered; and after assembly, the meshed tube is shaped into an cylinder.

3. The radiofrequency ablation catheter as claimed in claim 1, wherein before assembly, the meshed tube is shaped into an cylinder; and after assembly, the meshed tube is shaped into a round drum body which has an intermediate convex and both naturally tapered ends.

4. The radiofrequency ablation catheter as claimed in claim 1, further comprising:
a radiofrequency line and a thermocouple wire disposed inside each of the electrodes,
wherein the radiofrequency line, the thermocouple wire and the filament are independent wire materials; or a portion of the filament has a function of the radiofrequency line; or the radiofrequency line and the thermocouple wire are made into one wire.

5. The radiofrequency ablation catheter as claimed in claim 1, wherein axial projections of a plurality of the electrodes in an axial direction of the meshed tubular stent do not overlap each other.

6. The radiofrequency ablation catheter as claimed in claim 1, wherein a plurality of the electrodes are arranged in a straight line or staggered in a plurality of straight lines on an expansion diagram of a circumferential surface of the meshed tube.

7. The radiofrequency ablation catheter as claimed in claim 1, wherein the both ends of the meshed tube are provided with a first connecting tube and a second connecting tube; the meshed tubular stent further includes a central drawing filament disposed along a central axis thereof, wherein one end of the central drawing filament is fixed on the first connecting tube disposed at the distal end of the meshed tubular stent, or the central drawing filament penetrates through the first connecting tube and is confined outside the first connecting tube;
the other end of the central drawing filament penetrates through an inside of the meshed tubular stent and then through a center of the second connecting tube disposed at the proximal end of the meshed tubular stent; the central drawing filament is configured to axially draw the meshed tubular stent relative to the second connecting tube, and the central drawing filament is configured to slide toward the distal end of the meshed tubular stent relative to the second connecting tube.

8. The radiofrequency ablation catheter as claimed in claim 7, wherein the proximal end of the meshed tubular stent is connected to a multi-hole tube, wherein one end of the central drawing filament is fixed on the distal end of the meshed tubular stent, or the central drawing filament is confined outside the distal end of the meshed tubular stent, and thus configured to freely slide relative to the distal end of the meshed tubular stent; wherein the other end of the central drawing filament penetrates through a central hole of the multi-mole tube; a radiofrequency line, a thermocouple wire and the filament are disposed inside each of the electrodes; both ends of the electrodes are fixed on the meshed tubular stent; one end of the thermocouple wire and one end of the radiofrequency line are fixed inside the electrode; and the other end of the thermocouple wire and the other end of the radiofrequency line penetrate through a corresponding hole in the multi-hole tube and then are connected to an external device.

9. The radiofrequency ablation catheter as claimed in claim 1, wherein an opening is disposed on a circumference of each of the electrode.

10. The radiofrequency ablation catheter as claimed in claim 1, wherein the meshed tube is woven and formed by one single of the filament or a plurality of the filaments; or the meshed tube is processed and formed by a metal material or a polymer material.

11. A radiofrequency ablation apparatus, comprising a radiofrequency ablation catheter as claimed in claim 1, and a control handle and a radiofrequency ablation main machine, both connected to the radiofrequency ablation catheter.
